(19) United States
(12) Patent Application Publication
(10) Pub. No.: US 2015/0125447 A1
(43) Pub. Date: May 7, 2015

(54) PHARMACEUTICAL COMBINATIONS COMPRISING CD33 ANTIBODIES AND DE-METHYLATING AGENTS

(71) Applicant: Boehringer Ingelheim International GmbH, Ingelhaim am Rhein (DE)

(72) Inventor: Karl-Heinz HEIDER, Stockerau (AU)

(21) Appl. No.: 14/533,136

(22) Filed: Nov. 5, 2014

(30) Foreign Application Priority Data
Nov. 6, 2013 (EP) 13 191 839.3

(51) Int. Cl. A61K 39/395; A61K 31/706

(52) U.S. Cl. CPC A61K 39/39558; A61K 31/706; A61K 2039/572
USPC 424/133.1; 424/173.1

(57) ABSTRACT

The present invention relates to pharmaceutical combinations CD33 antibodies and de-methylating agents for use in treating diseases like MDS and cancer, especially AML.

### FIELD OF INVENTION

The present invention relates to pharmaceutical combinations of CD33 antibodies and de-methylating agents for use in treating diseases like MDS and cancer, especially AML.

### BACKGROUND OF INVENTION

In the early 1980s CD33 was identified as a marker of myeloid leukemias (Andrews et al., Blood 62, 24-132, 1983). CD33 is a cell-surface antigen specifically expressed on myeloid cells including myeloid leukemia cells. It is the smallest member of the siglec (sialic acid-binding Ig-related lectins) family. CD33 is expressed on early multilineage hematopoietic progenitor cells and myelomonocytic precursors. It is absent from pluripotent hematopoietic stem cells (Andrews et al., Journal of Experimental Medicine 169, 1721-1731, 1989). It is downregulated on mature granulocytes but retained on macrophages, monocytes and dendritic cells (Andrews et al., Blood 62, 24-132, 1983). Besides myelomonocytic cells, CD33 has also been found to be expressed on human mast cells and blood basophils (Valent et al., Blood 15; 73(7):1778-85, 1989). Monoclonal antibodies directed against CD33 are used in diagnosis of leukemia as well as for therapeutic targeting and in vitro purging of bone marrow for autologous transplantation in acute myeloid leukemia (AML) (Duzkale et al., Biol Blood Marrow Transplant. 9(6):364-72, 2003). Initial efforts in therapeutic targeting focused on the development of immunotoxins using an anti-CD33 antibody conjugated to the toxin ricin. CD33 rapidly internalizes upon antibody binding (Audran et al., J Immunol Methods. 188(1):147-54, 1995).

CD33 is a 67 KD transmembrane glycoprotein. The sialic acid-binding extracellular domain of CD33 is involved in cell-cell adhesion. The intracellular immunoreceptor tyrosine-based inhibitory motifs (ITIM) confer inhibitory signals to the cell, affecting proliferation and cell survival. The actual signalling pathways of CD33 are poorly understood but are assumed to involve the ITIM and ITIM-like motifs and the recruitment of tyrosine phosphatases (von Gunten et al., Ann. N.Y. Acad. Sci. 1143: 61-82, 2008). A murine CD33 orthologue has been defined but its functional comparability to human CD33 was questioned (Brinkman-Van der Linden et al., Mol Cell Biol., 23(12): 4199-206, 2003). The functional role of human CD33 on normal and malignant leukocytes remains unknown.

Several publications have described CD33 as a stable cell surface marker on primary AML and CML cells expressed by 70-100% of tested patients (Plesa et al., Cancer 112(3), 572-80, 2007; Hauswirt et al., Eur J Clin Invest. Jan. 73-82, 2007; Scheinberg et al., Leukemia Vol. 3, 440-445, 1989). CD33 is expressed on malignant myeloid blast cells, which represent the majority of malignant cells in peripheral blood and bone marrow of leukemia patients, and on leukemic stem cells, a relatively small number of less differentiated cells in the bone marrow which are characterized by their capacity for self-renewal and the maintenance of the leukemic clonal hierarchy. Depletion of leukemic stem cells is regarded the key mechanism for sustained tumor free survival. The CD33 targeting immunotoxin Mylotarg®, a humanized IgG4 antibody conjugated to the toxin calicheamicin is used for the treatment of AML patients by delivering its toxic payload to CD33 positive AML cells (Amadori et al., Cancer Treat Rev. 34(1):49-60, 2008). Lintuzumab (SGN-33, HuM195), a “naked” CD33 specific humanized monoclonal antibody was evaluated in phase II clinical trials for the treatment of AML and MDS with initial clinical signs of efficacy from a phase I dose escalation study and tolerable adverse events being reported (Raza et al. Abstract #983, 14th EHA Congress, Jun. 4-7, 2009).

Targeting AML cell lines with CD33 specific HuM195 in vitro reduces TNF-α induced secretion of inflammatory cytokines like IL-8, MCP-1 and RANTES (Sutherland et al., Mabs 1:5, 481-490, 2009). The relevance of this effect for AML therapy is unknown but modulating the cytokine milieu of the tumor microenvironment may contribute to the therapeutic efficacy of the antibody. In addition, the antibody induces antibody-dependent cell-mediated cytotoxicity (ADCC) and antibody-dependent cell-mediated phagocytosis (ADCP) of AML cell lines in vitro (Sutherland et al., Mabs 1:5, 481-490, 2009). ADCC is considered to be a decisive mechanism for anti-tumor activity of antibodies in hematological malignancies. Data from clinical trials with the CD20-specific monoclonal antibody Rituximab have demonstrated the significance of effector cell mediated mechanisms for treatment of B-cell malignancies with respect to response to antibody treatment (Weng and Levy, J Clin Oncol. 21 (21): 3940-7, 2003).

In conclusion, it has been shown that CD33 antigen is expressed on normal cells of the myelomonocytic lineage and frequently expressed on tumor cells in myeloid leukemias. In a phase I trial with an antibody against CD33 (lintuzumab) first signs of efficacy were observed without severe adverse events. However, clinical development of lintuzumab was discontinued after results from a phase II trial in combination with chemotherapy did not yield the expected improvement in efficacy. Therefore, there is a clear need for the development of improved CD33-targeting treatment modalities.

In view of the prior art there is a need for providing improved therapies for myeloid cell malignancies, particularly for acute myeloid leukemia and MDS.

### SUMMARY OF THE INVENTION

The present invention provides pharmaceutical combinations comprising CD33 antibodies and de-methylating agents for the treatment of cancer, especially acute myeloid leukemia (AML), and myelodysplastic syndrome (MDS).

The CD33 antibodies disclosed in the present application bind to CD33-expressing tumor cells in myeloid leukemias (especially AML) or to myeloid derived suppressor cells (MDSC) in the bone marrow of MDS patients and recruit effector cells from the circulation, which then destroy the CD33-expressing tumor cells or MDSCs. Moreover, the CD33 antibodies of concern here are preferably provided with certain mutations in the Fc part, which provide the antibodies with increased ADCC (antibody dependent cellular cytotoxicity) activity.

Treatment of myeloid leukemias, especially AML and of MDS with de-methylating agents, like 5-azacytidine (azacitidine) and 5-azadeoxycytidine (decitabine), SGI-110 is an established therapy in this field of cancer and myelodysplastic syndromes. De-methylating agents, in particular azacytidine are however known to decrease the activity of human effector cells, e.g. natural killer cells (Gao et al., Molecular Immunology 46 (2009) 2064-2070; to a significant extend. Therefore, it is considered a prejudice in the art that treating AML and MDS patients with de-methylating agents will be detrimenting the activity of their immune system effector cells, in particular of their NK cells. Therefore a person skilled in the art would expect that concomitant therapy with de-methylating agents and ADCC-mediating antibodies would be less effective than antibody monotherapy due to the inhibitory action of the de-methylating agent on NK cell activity which is required for the antibodies ADCC activity.

Unexpectedly evidence has now been found that a combination therapy of de-methylating agents and certain CD33 antibodies with enhanced ADCC activity does not result in lower ADCC activity of the antibodies. This unexpected result opens a new therapeutic approach for treating cancer diseases like AML and MDS with high efficacy.

### BRIEF DESCRIPTION OF THE FIGURES

FIG. 1 is a plot of the data shown in table 2 and illustrates the influence of the de-methylating agents on the ADCC activity of the CD33 antibody CD33-1.

### DETAILED DESCRIPTION OF THE INVENTION

“Pharmaceutical combinations” as used herein refer to two or more different pharmaceutically-active substances, which are intended to produce a specific therapeutic effect in a patient when applied together to said patient, i.e. one or more CD33 antibodies and one or more de-methylating agents in the context of the present invention. “Applied together” herein means either sequential application or simultaneous application.

In one embodiment, the CD33 antibody is to be administered at any time point between 6 months and 1 week prior to administration of the de-methylating agent. In preferred embodiments, the CD33 antibody is to be administered at any time point between 3 months and 1 week, six weeks and 1 week, 1 month and 1 week, 3 weeks and 1 week, and 2 weeks and 1 week prior to administration of the de-methylating agent. In one embodiment, the CD33 antibody is to be administered at any time point between 1 week and 0 days prior to administration of the de-methylating agent.

Of course, it is also within the scope of the invention that the de-methylating agent is administered prior to the CD33 antibody. Hence, the aforementioned embodiment applies to this alternative embodiment, mutatis mutandis.

The administration of the CD33 antibody concurrently with the de-methylating agent means that both medicaments are administered at the same time. This can be achieved by having both CD33 antibody and de-methylating agent present in one dose, vial, bag, container, syringe, etc.

A subsequent administration of the CD33 antibody and de-methylating agent means that the de-methylating agent is administered shortly after the CD33 antibodies or vice versa. Shortly includes 1, 2, 3, 4, 5, 10, 20, 30, 45, 60 minutes, 2, 3, 4, 5, 6, 7, 8, 9, 10, 12, 14, 16, 18, 20, 22 or 24 hours.

“Patient” herein refers to mammals, particularly humans.

“CD33 antibodies” as used herein refers to IgG1-type antibodies comprising two immunoglobulin heavy chains and two immunoglobulin light chains, which have immunospecificity for binding to CD33. Antibodies are generally described in, for example, Harlow and Lane, Antibodies: A Laboratory Manual (Cold Spring Harbor Laboratory Press, 1988). The CD33 antibodies may be unconjugated antibodies like lintuzumab (SGN-33), My9 or antibody-drug conjugates like Mylotag® or SGN-CD33A), or bi-specific CD33 T-cell or NK-cell engagers (e.g. CD33-BiTE or CD33-BiKE). Preferably the CD33 antibodies used herein are humanized, more preferably fully human. Preferred CD33 antibodies herein are provided with an effector function, i.e. an Fc domain. In preferred embodiments that Fc domain is provided with mutations that increase ADCC by at least 10%, preferably 50% and more preferably 100%. Preferably such mutations in the Fc domain are located at one or more positions selected from amino acids at positions 332 and/or 239 and/or 236 according to the Kabat EU numbering index. Preferably the mutations are S239D/I332E.

“Heavy chain variable region” or “VH” means the part of the heavy chain comprising the CDR1, CDR2 and CDR3 and surrounding framework regions.

“Light chain variable region” or “VL” means the part of the light chain comprising the CDR4, CDR5 and CDR6 and surrounding framework regions.

“CDR” means the hypervariable regions of the heavy and light chains, which determine the complementarity/binding specificity of an antibody or antibody fragment. The order of the CDRs in the present application is purely numerically.

“Epitope” herein means a part of an antigen, which is recognized by an antibody or antibody fragment. In particular this term refers to parts of CD33, which can be recognized by an antibody.

An antibody may have one or more “effector functions” which refer to those biological activities attributable to the Fc region (a native sequence Fc region or amino acid sequence variant Fc region) of an antibody. Examples of antibody effector functions include Clq binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc.

The following table (table 1) lists preferred antibodies under the invention and their constituents.

TABLE 1

| | | | | | | | | | | SeqID | SeqID |
|---|---|---|---|---|---|---|---|---|---|---|---|
| | Clone | SeqID | SeqID | SeqID | SeqID | SeqID | SeqID | SeqID | SeqID | heavy | light |
| No | ID# | CDR1 | CDR2 | CDR3 | CDR4 | CDR5 | CDR6 | V_{H} | V_{L} | chain | chain |
| 1 | 280- | 1 | 15 | 29 | 43 | 57 | 71 | 85 | 99 | 113 | 127 |
| | 03-08 | | | | | | | | | | |
| 2 | 280- | 2 | 16 | 30 | 44 | 58 | 72 | 86 | 100 | 114 | 128 |
| | 21-09 | | | | | | | | | | |
| 3 | 280- | 3 | 17 | 31 | 45 | 59 | 73 | 87 | 101 | 115 | 129 |
| | 29-12 | | | | | | | | | | |
| 4 | 280- | 4 | 18 | 32 | 46 | 60 | 74 | 88 | 102 | 116 | 130 |
| | 31-01 | | | | | | | | | | |
| 5 | 280- | 5 | 19 | 33 | 47 | 61 | 75 | 89 | 103 | 117 | 131 |
| | 31-01 | | | | | | | | | | |
| | (mut) | | | | | | | | | | |
| 6 | 280- | 6 | 20 | 34 | 48 | 62 | 76 | 90 | 104 | 118 | 132 |
| | 34-02 | | | | | | | | | | |
| 7 | 280- | 7 | 21 | 35 | 49 | 63 | 77 | 91 | 105 | 119 | 133 |
| | 50-01 | | | | | | | | | | |
| 8 | 280- | 8 | 22 | 36 | 50 | 64 | 78 | 92 | 106 | 120 | 134 |
| | 50-01 | | | | | | | | | | |
| | (mut) | | | | | | | | | | |
| 9 | 280- | 9 | 23 | 37 | 51 | 65 | 79 | 93 | 107 | 121 | 135 |
| | 61-07 | | | | | | | | | | |
| 10 | 283- | 10 | 24 | 38 | 52 | 66 | 80 | 94 | 108 | 122 | 136 |
| | 03-03 | | | | | | | | | | |
| 11 | 283- | 11 | 25 | 39 | 53 | 67 | 81 | 95 | 109 | 123 | 137 |
| | 05-01 | | | | | | | | | | |
| 12 | 283- | 12 | 26 | 40 | 54 | 68 | 82 | 96 | 110 | 124 | 138 |
| | 07-03 | | | | | | | | | | |
| 13 | 283- | 13 | 27 | 41 | 55 | 69 | 83 | 97 | 111 | 125 | 139 |
| | 11-03 | | | | | | | | | | |
| 14 | 283- | 14 | 28 | 42 | 56 | 70 | 84 | 98 | 112 | 126 | 140 |
| | 14-01 | | | | | | | | | | |

Particularly preferred CD33 antibodies are selected from

an antibody comprising a CDR1 of SeqID No: 1, a CDR2 of SeqID No: 15, a CDR3 of SeqID No: 29, a CDR4 of SeqID No: 43, a CDR5 of SeqID No: 57 and a CDR6 of SeqID No: 71,
an antibody comprising a CDR1 of SeqID No: 2, a CDR2 of SeqID No: 16, a CDR3 of SeqID No: 30, a CDR4 of SeqID No: 44, a CDR5 of SeqID No: 58 and a CDR6 of SeqID No: 72,
an antibody comprising a CDR1 of SeqID No: 3, a CDR2 of SeqID No: 17, a CDR3 of SeqID No: 31, a CDR4 of SeqID No: 45, a CDR5 of SeqID No: 59 and a CDR6 of SeqID No: 73,
an antibody comprising a CDR1 of SeqID No: 4, a CDR2 of SeqID No: 18, a CDR3 of SeqID No: 32, a CDR4 of SeqID No: 46, a CDR5 of SeqID No: 60 and a CDR6 of SeqID No: 74,
an antibody comprising a CDR1 of SeqID No: 5, a CDR2 of SeqID No: 19, a CDR3 of SeqID No: 33, a CDR4 of SeqID No: 47, a CDR5 of SeqID No: 61 and a CDR6 of SeqID No: 75,
an antibody comprising a CDR1 of SeqID No: 6, a CDR2 of SeqID No: 20, a CDR3 of SeqID No: 34, a CDR4 of SeqID No: 48, a CDR5 of SeqID No: 62 and a CDR6 of SeqID No: 76,
an antibody comprising a CDR1 of SeqID No: 7, a CDR2 of SeqID No: 21, a CDR3 of SeqID No: 35, a CDR4 of SeqID No: 49, a CDR5 of SeqID No: 63 and a CDR6 of SeqID No: 77,
an antibody comprising a CDR1 of SeqID No: 8, a CDR2 of SeqID No: 22, a CDR3 of SeqID No: 36, a CDR4 of SeqID No: 50, a CDR5 of SeqID No: 64 and a CDR6 of SeqID No: 78,
an antibody comprising a CDR1 of SeqID No: 9, a CDR2 of SeqID No: 23, a CDR3 of SeqID No: 37, a CDR4 of SeqID No: 51, a CDR5 of SeqID No: 65 and a CDR6 of SeqID No: 79,
an antibody comprising a CDR1 of SeqID No: 10, a CDR2 of SeqID No: 24, a CDR3 of SeqID No: 38, a CDR4 of SeqID No: 52, a CDR5 of SeqID No: 66 and a CDR6 of SeqID No: 80,
an antibody comprising a CDR1 of SeqID No: 11, a CDR2 of SeqID No: 25, a CDR3 of SeqID No: 39, a CDR4 of SeqID No: 53, a CDR5 of SeqID No: 67 and a CDR6 of SeqID No: 81,
an antibody comprising a CDR1 of SeqID No: 12, a CDR2 of SeqID No: 26, a CDR3 of SeqID No: 40, a CDR4 of SeqID No: 54, a CDR5 of SeqID No: 68 and a CDR6 of SeqID No: 82,
an antibody comprising a CDR1 of SeqID No: 13, a CDR2 of SeqID No: 27, a CDR3 of SeqID No: 41, a CDR4 of SeqID No: 55, a CDR5 of SeqID No: 69 and a CDR6 of SeqID No: 83,
an antibody comprising a CDR1 of SeqID No: 14, a CDR2 of SeqID No: 28, a CDR3 of SeqID No: 42, a CDR4 of SeqID No: 56, a CDR5 of SeqID No: 70 and a CDR6 of SeqID No: 84.

Particularly preferred CD33 antibodies are selected from

an antibody comprising a heavy chain variable region of SeqID No: 85 and a light chain variable region of SeqID No: 99,
an antibody comprising a heavy chain variable region of SeqID No: 86 and a light chain variable region of SeqID No: 100,
an antibody comprising a heavy chain variable region of SeqID No: 87 and a light chain variable region of SeqID No: 101,
an antibody comprising a heavy chain variable region of SeqID No: 88 and a light chain variable region of SeqID No: 102,
an antibody comprising a heavy chain variable region of SeqID No: 89 and a light chain variable region of SeqID No: 103,
an antibody comprising a heavy chain variable region of SeqID No: 90 and a light chain variable region of SeqID No: 104,
an antibody comprising a heavy chain variable region of SeqID No: 91 and a light chain variable region of SeqID No: 105,
an antibody comprising a heavy chain variable region of SeqID No: 92 and a light chain variable region of SeqID No: 106,
an antibody comprising a heavy chain variable region of SeqID No: 93 and a light chain variable region of SeqID No: 107,
an antibody comprising a heavy chain variable region of SeqID No: 94 and a light chain variable region of SeqID No: 108,
an antibody comprising a heavy chain variable region of SeqID No: 95 and a light chain variable region of SeqID No: 109,
an antibody comprising a heavy chain variable region of SeqID No: 96 and a light chain variable region of SeqID No: 110,
an antibody comprising a heavy chain variable region of SeqID No: 97 and a light chain variable region of SeqID No: 111,
an antibody comprising a heavy chain variable region of SeqID No: 98 and a light chain variable region of SeqID No: 113,

Particularly preferred CD33 antibodies are selected from

an antibody comprising a heavy chain of SeqID No: 113 and a light chain of SeqID No: 127,
an antibody comprising a heavy chain of SeqID No: 114 and a light chain of SeqID No: 128,
an antibody comprising a heavy chain of SeqID No: 115 and a light chain of SeqID No: 129,
an antibody comprising a heavy chain of SeqID No: 116 and a light chain of SeqID No: 130,
an antibody comprising a heavy chain of SeqID No: 117 and a light chain of SeqID No: 131,
an antibody comprising a heavy chain of SeqID No: 118 and a light chain of SeqID No: 132,
an antibody comprising a heavy chain of SeqID No: 119 and a light chain of SeqID No: 133,
an antibody comprising a heavy chain of SeqID No: 120 and a light chain of SeqID No: 134,
an antibody comprising a heavy chain of SeqID No: 121 and a light chain of SeqID No: 135,
an antibody comprising a heavy chain of SeqID No: 122 and a light chain of SeqID No: 136,
an antibody comprising a heavy chain of SeqID No: 123 and a light chain of SeqID No: 137,
an antibody comprising a heavy chain of SeqID No: 124 and a light chain of SeqID No: 138,
an antibody comprising a heavy chain of SeqID No: 125 and a light chain of SeqID No: 139,
an antibody comprising a heavy chain of SeqID No: 126 and a light chain of SeqID No: 140.

Preferred CD33 antibodies herein are those disclosed by the applicant's patent application WO 2012/045752, i.e. CD33 antibodies recognizing an epitope within the amino acid sequence FFHPIPYYDKNSPVHGYW (SeqID No: 141, determined by Hydrogen Exchange Spectroscopy) of human CD33.

“CD33-1” is a CD33 antibody according to example No. 5 in table 1 herein, having an immunoglobulin heavy chain according to SeqID No: 117 and an immunoglobulin light chain according to SeqID No: 131.

“De-methylating agents” as used herein refers to pharmacologically acceptable inhibitors of DNA methyl transferase. De-methylating agents cause hypomethylation of the DNA. The term demethylating agents refers to a group of chemotherapeutic agents with the capacity, both in vitro and in vivo, to induce transient DNA hypomethylation. DNA methylation refers to the addition of a methyl group to a CpG site (G. Garcia-Manero, Current Opinion in Oncology 2008, 20:705-710). These sites cluster together in areas known as CpG islands and are frequently localized in the proximity of key gene regulatory regions such as gene promoters. DNA methylation, both aberrant and physiologic, of these areas can result in gene silencing and in the equivalent of the physical inactivation, due to either mutations or deletions, of tumor suppressor genes. Two hypomethylating agents are approved in the United States and are widely used in Europe and the rest of the world: 5-azacitidine and 5-aza-2′-deoxycitidine (decitabine) (Silverman et al., B. J Clin Oncol 2002; 20:2429-2440; Kantarjian et al., Cancer 2003; 98: 522 528). Another de-methylating agent is SGI-110, which is currently developed by Astex Pharmaceuticals.

“Decitabine” as used herein refers to 4-Amino-1-(2-deoxy-β-D-erythro-pentofuranosyl)-1,3,5-triazin-2(1H)-one, which is sold e.g. under the trade name Dacogen®. Decitabine is also referred to as 5-azadeoxycytidine.

“5-azacytidine” as used herein refers to 4-amino-1-β-D-ribofuranosyl-1,3,5-triazin-2(1H)-one, as soled e.g. under the trade name Vidaza®. 5-azacytidine is also referred to as azacitidine.

“SGI-110” as used herein refers to (2R,3S,5R)-5-(4-amino-2-oxo-1,3,5-triazin-1(2H)-yl)-2-(hydroxymethyl)tetrahydrofuran-3-yl (((2S,3R,5R)-5-(2-amino-6-oxo-1H-purin-9(6H)-yl)-3-hydroxytetrahydrofuran-2-yl)methyl) hydrogen phosphate as currently developed by Astex Pharmaceuticals.

“Cancer” as used herein generally to all malignant neoplastic diseases. For example, the following cancers may be treated with combinations according to the invention, without being restricted thereto:

brain tumours such as for example acoustic neurinoma, astrocytomas such as pilocytic astrocytomas, fibrillary astrocytoma, protoplasmic astrocytoma, gem istiocytic astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningiomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma. Bone marrow tumours; intestinal cancer such as for example carcinoma of the rectum and colon tumours of the small intestine and duodenum; esophageal cancer or cancer of the esophagus such as squamous cell carcinoma, adenocarcinoma in Barret's esophagus, adenoid cystic carcinoma, small cell carcinoma and lymphoma; eyelid tumours such as basalioma or basal cell carcinoma; pancreatic cancer or carcinoma of the pancreas such as duct cell adenocarcinoma, acinar cell carcinoma, islet cell carcinoma, lymphoma and sarcoma of the pancreas; bladder cancer or carcinoma of the bladder such as superficial and infiltrating transitional cell carcinoma, squamous cell carcinoma and adenocarcinoma; lung cancer (bronchial carcinoma) such as for example small-cell bronchial carcinomas (oat cell carcinomas) and non-small cell bronchial carcinomas (NSCLC) such as squamous cell carcinomas, adenocarcinomas and large-cell bronchial carcinomas; breast cancer such as for example mammary carcinoma such as in situ and infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrioid and serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supraglottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma such as for example clear cell renal cell carcinoma or hypernephroma or Grawitz's tumour, papillary carcinoma and oncocytoma; oesophageal cancer or carcinoma of the oesophagus; penile cancer; prostate cancer; throat cancer or carcinomas of the pharynx such as for example squamous cell carcinomas of the nasopharynx (nasopharynx carcinomas), oropharynx (oropharynx carcinomas) and hypopharynx carcinomas; retinoblastoma, vagin cancer or vaginal carcinoma and cancers of the vulva including squamous cell carcinomas, adenocarcinomas and in situ carcinomas; malignant melanomas and sarcomas; thyroid carcinomas such as for example papillary, follicular and medullary thyroid carcinoma, as well as anaplastic carcinomas; spinalioma, epiderrmoid carcinoma and basal cell carcinoma of the skin; thymomas, cancer of the urethra including in situ and infiltrating transitional cell carcinoma.

Preferred are cancers, which express CD33 on the surface of the tumor cells.

“MDS” herein refers to myelodysplastic syndrome, formerly known as preleukemia.

Combinations with Anti-Neoplastic Agents:

In preferred embodiments of the invention the pharmaceutical combinations herein further comprise one or more “anti-neoplastic agents”, which term is used herein to refer to a substance producing an anti-neoplastic effect in a tissue, system, animal, mammal, human, or other subject. In particular, in anti-neoplastic therapy, combination therapy with other chemotherapeutic, hormonal, antibody agents as well as surgical and/or radiation treatments other than those mentioned above are envisaged. Combination therapies according to the present invention thus include the administration of CD33 antibodies and de-methylating agents as well as optional use of other therapeutic agents including other anti-neoplastic agents. Such combination of agents may be administered together or separately and, when administered separately this may occur simultaneously or sequentially in any order, both close and remote in time.

Depending on the disorder to be treated, the pharmaceutical combinations herein of the invention may be used on its own or in combination with one or more anti-neoplastic agents, in particular selected from DNA damaging, tubulin binding agents or therapeutically active compounds that inhibit angiogenesis, signal transduction pathways or mitotic checkpoints in cancer cells or have immunomodulatory function (IMIDs).

The anti-neoplastic agent may be administered simultaneously with, optionally as a component of the same pharmaceutical composition, or before or after administration of the pharmaceutical combinations herein.

In certain embodiments, the anti-neoplastic agent may be, without limitation, one or more inhibitors selected from the group of inhibitors of EGFR family, VEGF family, Angiopoietin family, CD37, IGF1 and 2, DII4, VEGF-R family, IGF-1R, Insulin receptors, AuroraA, AuroraB, PLK and PI3 kinase, FGFR, PDGFR, Raf, KSP or PDK1.

Further examples of anti-neoplastic agents are inhibitors of CDKs, Akt, Src, Bcr-Abl, cKit, cMet/HGF, Her2, Her3, c-Myc, Flt3, HSP90, hedgehog antagonists, inhibitors of JAK/STAT, Mek, mTor, NFkappaB, the proteasome, Rho, an inhibitor of Wnt signaling or Notch signaling or an ubiquitination pathway inhibitor.

Further examples of anti-neoplastic agents are inhibitors of DNA polymerase, topoisomerase II, multityrosine kinase inhibitors, CXCR4 antagonists, IL3RA inhibitors, RAR antagonists, KIR inhibitors, immunotherapeutic vaccines, TUB inhibitors, Hsp70 inducers, IAP family inhibitors, DNA methyltransferase inhibitors, TNF inhibitors, ErbB1 receptor tyrosine kinase inhibitors, multikinase inhibitors, JAK2 inhibitors, RR inhibitors, apoptosis inducers, HGPRTase inhibitors, histamine H2 receptor antagonists and CD25 receptor agnosists.

Examples for Aurora inhibitors are, without limitation, PHA-739358, AZD-1152, AT-9283, CYC-116, R-763, VX-667, MLN-8045, PF-3814735, SNS-314, VX-689, GSK-1070916, TTP-607, PHA-680626, MLN-8237, BI847325 and ENMD-2076.

Examples for PLK inhibitor are GSK-461364, BI2536 and BI6727.

Examples for raf inhibitors are BAY-73-4506 (also a VEGF-R inhibitor), PLX-4032, RAF-265 (also a VEGF-R inhibitor), sorafenib (also a VEGF-R inhibitor), XL-281, Nevavar (also an inhibitor of the VEGF-R) and PLX4032.

Examples for KSP inhibitors are ispinesib, ARRY-520, AZD-4877, CK-1122697, GSK-246053A, GSK-923295, MK-0731, SB-743921, LY-2523355, and EMD-534085.

Examples for a src and/or bcr-abl inhibitors are dasatinib, AZD-0530, bosutinib, XL-228 (also an IGF-1R inhibitor), nilotinib (also a PDGFR and cKit inhibitor), imatinib (also a cKit inhibitor), NS-187, KX2-391, AP-24534 (also an inhibitor of EGFR, FGFR, Tie2, Flt3), KM-80 and LS-104 (also an inhibitor of Flt3, Jak2).

An example for a PDK1 inhibitor is AR-12.

An example for a Rho inhibitor is BA-210.

Examples for PI3 kinase inhibitors are Idelalisib (Cal-101), PX-866, PX-867, BEZ-235 (also an mTor inhibitor), XL-147, and XL-765 (also an mTor inhibitor), BGT-226, CDC-0941.

Examples for inhibitors of cMet or HGF are XL-184 (also an inhibitor of VEGF-R, cKit, Flt3), PF-2341066, MK-2461, XL-880 (also an inhibitor of VEGF-R), MGCD-265 (also an inhibitor of VEGF-R, Ron, Tie2), SU-11274, PHA-665752, AMG-102, AV-299, ARQ-197, MetMAb, CGEN-241, BMS-777607, JNJ-38877605, PF-4217903, SGX-126, CEP-17940, AMG-458, INCB-028060, and E-7050.

An example for a Notch pathway inhibitor is MEGF0444A.

An example for a c-Myc inhibitor is CX-3543.

Examples for Flt3 inhibitors are AC-220 (also an inhibitor of cKit and PDGFR), KW-2449, LS-104 (also an inhibitor of bcr-abl and Jak2), MC-2002, SB-1317, lestaurtinib (also an inhibitor of VEGF-R, PDGFR, PKC), TG-101348 (also an inhibitor of JAK2), XL-999 (also an inhibitor of cKit, FGFR, PDGFR and VEGF-R), sunitinib (also an inhibitor of PDGFR, VEGF-R and cKit), and tandutinib (also an inhibitor of PDGFR, and cKit).

Examples for HSP90 inhibitors are tanespimycin, alvespimycin, IPI-504, STA-9090, MEDI-561, AUY-922, CNF-2024, and SNX-5422.

Examples for JAK/STAT inhibitors are CYT-997 (also interacting with tubulin), TG-101348 (also an inhibitor of Flt3), and XL-019.

Examples for Mek inhibitors are ARRY-142886, AS-703026, PD-325901, AZD-8330, ARRY-704, RDEA-119, and XL-518.

Examples for mTor inhibitors are temsirolimus, deforolimus (which also acts as a VEGF inhibitor), everolimus (a VEGF inhibitor in addition), XL-765 (also a PI3 kinase inhibitor), and BEZ-235 (also a PI3 kinase inhibitor).

Examples for Akt inhibitors are perifosine, GSK-690693, RX-0201, and triciribine.

Examples for cKit inhibitors are masitinib, OSI-930 (also acts as a VEGF-R inhibitor), AC-220 (also an inhibitor of Flt3 and PDGFR), tandutinib (also an inhibitor of Flt3 and PDGFR), axitinib (also an inhibitor of VEGF-R and PDGFR), sunitinib (also an inhibitor of Flt3, PDGFR, VEGF-R), and XL-820 (also acts as a VEGF-R- and PDGFR inhibitor), imatinib (also a bcr-abl inhibitor), nilotinib (also an inhibitor of bcr-abl and PDGFR).

Examples for hedgehog antagonists are IPI-609, CUR-61414, GDC-0449, IPI-926, and XL-139.

Examples for CDK inhibitors are seliciclib, AT-7519, P-276, ZK-CDK (also inhibiting VEGF-R2 and PDGFR), PD-332991, R-547, SNS-032, PHA-690509, PHA-848125, and SCH-727965.

Examples for proteasome inhibitors are bortezomib, carfilzomib, and NPI-0052 (also an inhibitor of NFkappaB).

Examples for proteasome inhibitors/NFkappaB pathway inhibitors are bortezomib, carfilzomib, NPI-0052, CEP-18770, MLN-2238, PR-047, PR-957, AVE-8680, and SPC-839.

An example for an inhibitor of the ubiquitination pathway is HBX-41108.

Examples for demethylating agends are 5-azacitidine and decitabine.

Examples for anti-angiogenic agents are inhibitors of the FGFR, PDGFR and VEGF, and thalidomides, such agents being selected from, without limitation, olaratumab, pegdinetanib, motesanib, CDP-791, SU-14813, telatinib, KRN-951, ZK-CDK (also an inhibitor of CDK), ABT-869, BMS-690514, RAF-265, IMC-KDR, IMC-18F1, IMiDs, thalidomide, CC-4047, lenalidomide, ENMD-0995, IMC-D11, Ki-23057, brivanib, cediranib, 1B3, CP-868596, IMC-3G3, R-1530 (also an inhibitor of Flt3), sunitinib (also an inhibitor of cKit and Flt3), axitinib (also an inhibitor of cKit), lestaurtinib (also an inhibitor of Flt3 and PKC), vatalanib, tandutinib (also an inhibitor of Flt3 and cKit), pazopanib, PF-337210, E-7080, CHIR-258, sorafenib tosylate (also an inhibitor of Raf), vandetanib, CP-547632, OSI-930, AEE-788 (also an inhibitor of EGFR and Her2), BAY-57-9352 (also an inhibitor of Raf), BAY-73-4506 (also an inhibitor of Raf), XL-880 (also an inhibitor of cMet), XL-647 (also an inhibitor of EGFR and EphB4), XL-820 (also an inhibitor of cKit), nilotinib (also an inhibitor of cKit and brc-abl), CYT-116, PTC-299, BMS-584622, CEP-11981, dovitinib, CY-2401401, ENMD-2976, ramucirumab, pegdinetanib and BIBF1120.

The anti-neoplastic agent may also be selected from EGFR inhibitors, it may be a small molecule EGFR inhibitor or an anti-EGFR antibody. Examples for anti-EGFR antibodies, without limitation, are cetuximab, panitumumab, nimotuzumab, zalutumumab; examples for small molecule EGFR inhibitors are gefitinib, erlotinib, vandetanib (also an inhibitor of the VEGF-R) and afatinib (also an inhibitor of Her2). Another example for an EGFR modulator is the EGF fusion toxin.

Further EGFR and/or Her2 inhibitors useful for combination with an Pharmaceutical combinations herein of the invention are lapatinib, trastuzumab, pertuzumab, XL-647, neratinib, BMS-599626 ARRY-334543, AV-412, mAB-806, BMS-690514, JNJ-26483327, AEE-788 (also an inhibitor of VEGF-R), AZD-8931, ARRY-380 ARRY-333786, IMC-11F8, Zemab, TAK-285, AZD-4769, and afatinib (dual inhibitor of Her2 and EGFR).

DNA polymerase inhibitors useful in the combination with pharmaceutical combinations herein are Ara-C/cytarabine, Clolar/clofarabine.

An apoptosis inducer useful in the combination with pharmaceutical combinations herein is Trisenox/arsenice trioxide.

Topoisomerase II inhibitors useful in the combination with pharmaceutical combinations herein are idarubicin, daunorubicin and mitoxantrone.

A RAR antagonist useful in the combination with pharmaceutical combinations herein is Vesanoid/tretinoin.

A HGPRTase inhibitor useful in the combination with pharmaceutical combinations herein is Mercapto/mercaptopurine.

A histamine H2 receptor antagonist useful in the combination with pharmaceutical combinations herein is Ceplene/histamine dihydrochloride.

A CD25 receptor agonist useful in the combination with pharmaceutical combinations herein is IL-2.

The anti-neoplastic agent may also be selected from agents that target the IGF-1R and insulin receptor pathways. Such agents include antibodies that bind to IGF-1R (e.g. CP-751871, AMG-479, IMC-A12, MK-0646, AVE-1642, R-1507, BIIB-022, SCH-717454, rhu Mab IGFR) and novel chemical entities that target the kinase domain of the IGF1-R (e.g. OSI-906 or BMS-554417, XL-228, BMS-754807).

Other anti-neoplastic agents that may be advantageously combined in a therapy with the pharmaceutical combinations herein of the invention are molecules targeting CD20, including CD20 specific antibodies like rituximab, LY-2469298, ocrelizumab, MEDI-552, IMMU-106, GA-101 (=R7159), XmAb-0367, ofatumumab, radiolabeled CD20 antibodies, like tositumumab and ibritumomab tiuxetan or other CD20 directed proteins, like the SMIP Tru015, PRO-131921, FBT-A05, veltuzumab, R-7159.

Pharmaceutical combinations herein may be combined with inhibitors of other surface antigens expressed on leukocytes, in particular antibodies or antibody-like molecules, e.g. anti-CD2 (siplizumab), anti-CD4 (zanolimumab), anti-CD19 (MT-103, MDX-1342, SAR-3419, XmAb-5574), anti-CD22 (epratuzumab), anti-CD23 (lumiliximab), anti-CD30 (iratumumab), anti-CD32B (MGA-321), anti-CD38 (HuMax-CD38), anti-CD40 (SGN40), anti-CD52 (alemtuzumab), anti-CD80 (galiximab).

Examples for suitable CD37 antibodies are BI836826, SMIP Tru016.

Suitable anti-VEGF agents are Avastin, Lucentis.

Other agents to be combined with pharmaceutical combinations herein are immunotoxins like BL-22 (an anti-CD22 immunotoxin), inotuzumab ozogamicin (an anti-CD23 antibody-calicheamicin conjugate), RFT5.dgA (anti-CD25 Ricin toxin A-chain), SGN-35 (an anti-CD30-auristatin E conjugate), and gemtuzumab ozogamicin (an anti-CD33 calicheamicin conjugate), MDX-1411 (anti-CD70 conjugate), or radiolabelled antibodies like ⁹⁰Y-epratuzumab (anti-CD22 radioimmunoconjugate).

In addition, pharmaceutical combinations herein may be combined with immunomodulators, agents, e.g. antibodies, that induce apoptosis or modify signal transduction pathways like the TRAIL receptor modulators mapatumumab (a TRAIL-1 receptor agonist), lexatumumab (a TRAIL-2 receptor agonist), tigatuzumab, Apomab, AMG-951 and AMG-655; an anti-HLA-DR antibody (like 1 D09C3), an anti-CD74, an osteoclast differentiation factor ligand inhibitor (like denosumab), a BAFF antagonist (like AMG-623a) or an agonist of a Toll-like receptor (e.g. TLR-4 or TLR-9).

Other anti-neoplastic agents that may be used in combination with the pharmaceutical combinations herein of the present invention are selected from, but not limited to hormones, hormonal analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortinsone, fluoxymesterone, medroxyprogesterone, hydroxyprogesterone caproate, diethylstilbestrol, testosterone propionate, fluoxymesterone/equivalents, octreotide, arzoxifene, pasireotide, vapreotide, adrenocorticosteroids/antagonists, prednisone, dexamethasone, ainoglutethimide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, trimetrexate, pemetrexed, pyrimidine analogues like 5-fluorouracil, fluorodeoxyuridine, capecitabine, decitabine, nelarabine, 5-azacytidine, and gemcitabine, purine and adenosine analogues such as mercaptopurine, thioguanine, azathioprine, cladribine and pentostatin, cytarabine, fludarabine, clofarabine); antitumor antibiotics (e.g. anthracyclines like doxorubicin, daunorubicin, epirubicin and idarubicin, mitomycin-C, bleomycin dactinomycin, plicamycin, splicamycin, actimomycin D, mitoxantrone, mitoxantroneidarubicin, pixantrone, streptozocin, aphidicolin); platinum derivatives (e.g. cisplatin, oxaliplatin, carboplatin, lobaplatin, satraplatin); alkylating agents (e.g. estramustine, semustine, mechlorethamine, melphalan, chlorambucil, busulphan, dacarbazine, cyclophosphamide, ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan, banoxantrone, camptothecin) and miscellaneous chemotherapeutics such as retinoic acid derivatives, amifostine, anagrelide, interferon alpha, interferon beta, interferon gamma, interleukin-2, procarbazine, N-methylhydrazine, mitotane, and porfimer, bexarotene, celecoxib, ethylenemine/methyl-melamine, thriethyienemelamine, triethylene thiophosphoramide, hexamethylmelamine, and enzymes L-asparaginase, L-arginase and metronidazole, misonidazole, desmethylmisonidazole, pimonidazole, etanidazole, nimorazole, RSU 1069, EO9, RB 6145, SR4233, nicotinamide, 5-bromodeozyuridine, 5-iododeoxyuridine, bromodeoxycytidine, erythrohydroxynonyl-adenine, anthracenedione, GRN-163L (a competitive telomerase template antagonist), SDX-101 (a PPAR agonist), talabostat (a DPP inhibitor), forodesine (a PNP inhibitor), atacicept (a soluble receptor targeting TNF family members BLyS and APRIL), TNF-alpha neutralizing agents (Enbrel, Humira, Remicade), XL-844 (a CHK1/2 inhibitor), VNP-40101M (a DNA alkylating agent), SPC-2996 (an antisense bcl2 inhibitor), obatoclax (a bcl2 inhibitor), enzastaurin (a PKC beta modulator), vorinistat (an HDAC inhibitor), romidepsin (an HDAC inhibitor), AT-101 (a Bcl-2/Bcl-xL inhibitor), plitidepsin (a multi-actioned depsipeptide), SL-11047 (a polyamine metabolism modulators).

The pharmaceutical combinations herein of the invention may also be used in combination with other therapies including surgery, stem cell transplantation, radiotherapy, endocrine therapy, biologic response modifiers, hyperthermia and cryotherapy and agents to attenuate any adverse effect (e.g. antiemetics), G-CSF, GM-CSF, photosensitizers such as hematoporphyrin derivatives, Photofrin, benzoporphyrin derivatives, Npe6, tin etioporphyrin, pheoboride-a bacteriochlorophyll-a, naphthalocyanines, phthalocyanines, zinc phthalocyanines.

Particularly preferred combinations partners are:

AML vaccine NBE JVRS-100,
Aminopeptidase inhibitor tosedostat,
Apoptosis inducer elesclomol,
Aurora kinase inhibitors AMG-900, ilorasertib, alisertib,
BRD (pan) inhibitor OTX-015,
CD33 antibody-drug conjugate SGN-CD33A,
CD44 antibody RG-7356,
Cell cycle checkpoint inhibitors DFP-10917; SCH-900776,
CRM1 nuclear export inhibitor KPT-330,
CXCR4 inhibitor MDX-1338; BL-8040 (BKT140),
Cytotoxic chemotherapy: OXi-4503; lurbinectedin; F-14512; vosaroxin; elacytarabine temozolomide; clofarabine; sapacitabine,

### Vaccine: DCP-001,

Eph A3 antibody: KB-004,
EGFR inhibitors erlotinib, afatinib,
FLT3 inhibitors AKN-028; sunitinib; crenolanib,
Grb2 antisense: BP-100.1.01,
HDAC inhibitors 4SC-202; panobinostat; entinostat, pracinostat, belinostat,
HDM inhibitors MK-8242,
Hedgehog inhibitors erismodegib; PF-04449913,
HSP inhibitor ganetespib,
IAP activators birinapant; Debio-1143 (AT-406),
IL-3 receptor antibody: CSL-362,
Immunomodulators lenalidomide; Ceplene,

### Immuno-toxin: SL-401,

IDH inhibitor AG-221,
JAK inhibitor ruxolitinib,
KIR antibody: lirilumab,
MDM2 inhibitor RO-5503781,
MEK inhibitors trametinib; pimasertib,
Mitotic kinesin inhibitor ARQ-621,
Multikinase inhibitors NCE PLX-3397; SB-1317; sorafenib; midostaurin,
Nedd 8 activating enzyme inhibitor pevonedistat (MLN-4924),
PARP inhibitor BMN-673,
PI3K/mTOR inhibitor BEZ-235,
PIM kinase inhibitor AZD1208,
PLK1 inhibitors rigosertib; volasertib,
Protein translation inhibitors omacetaxine, mepesuccinate,
Radio-immuno conjugate: lintuzumab-Ac225 (CD33),
src kinase inhibitors KX-01, (KX2-391),

### TPA PD-616,

Tumour activated NK cells: CNDO-109,
WNT pathway inhibitors PRI-724, CWP-232291,
WT-1 vaccines GSK-2130579A; OCV-501; FPI-01.

### Pharmaceutical Compositions and Methods of Administration

“Pharmaceutical composition” as used herein refers to a means to make the pharmaceutical combinations herein administrable to a patient. This means that the pharmaceutical combination as active ingredients of the pharmaceutical composition is admixed with one or more pharmaceutically acceptable diluents and optionally further pharmaceutically acceptable agents. The pharmaceutical composition herein can be in any form that allows for the pharmaceutical composition to be administered to a patient. For example, the pharmaceutical composition can be in the form of a solid or liquid. The preferred mode of application is parenteral, by infusion or injection (intravenous, intramuscular, subcutaneous, intraperitoneal, intradermal), but other modes of application such as by inhalation, transdermal, intranasal, buccal, oral and intra-tumor may also be applicable. Parenteral administration includes subcutaneous injections, intravenous, intramuscular, intrastemal injection or infusion techniques. In one aspect, the pharmaceutical compositions are administered parenterally. In yet another aspect, the pharmaceutical compositions are administered intravenously.

Pharmaceutical compositions can be formulated so as to allow a compound to be bioavailable upon administration of the pharmaceutical composition to a patient. Pharmaceutical compositions can take the form of one or more dosage units, where, for example, a container of a compound in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of patient (e.g., human), the particular form of the active constituents (i.e. CD33 antibodies and de-methylating agents, optionally anti-neoplastic agents), the manner of administration, and the pharmaceutical composition employed.

The pharmaceutically acceptable carrier or vehicle can be particulate, so that the pharmaceutical compositions are, for example, in powder form. The carrier(s) can be liquid, with the pharmaceutical compositions being, for example, an injectable liquid. The pharmaceutical composition can be in the form of a liquid, e.g., for parenteral injection. In a pharmaceutical composition for administration by injection, one or more of a surfactant, preservative, wetting agent, dispersing agent, suspending agent, buffer, stabilizer and isotonic agent can also be included.

The liquid pharmaceutical compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono- or diglycerides which can serve as the solvent or suspending medium, polyethylene glycols, glycerin, cyclodextrin, propylene glycol or other solvents; stabilizers such as amino acids; surfactants such as polysorbates; antibacterial agents such as benzyl alcohol or methyl paraben; antioxidants such as ascorbic acid or sodium bisulfite; chelating agents such as ethylenediaminetetraacelic acid; buffers such as acetates, citrates or phosphates; and agents for the adjustment of tonicity such as sodium chloride or dextrose. A parenteral pharmaceutical composition can be enclosed in ampoule, a disposable syringe or a multiple-dose vial made of glass, plastic or other material. Physiological saline is an exemplary adjuvant. An injectable pharmaceutical composition is preferably sterile.

The pharmaceutical compositions herein may also be dried (freeze-dried, spray-dried, spray-freeze dried, dried by near or supercritical gases, vacuum dried, air-dried), precipitated or crystallized or entrapped in microcapsules that are prepared, for example, by coacervation techniques or by interfacial polymerization using, for example, hydroxymethylcellulose or gelatin and poly-(methylmethacylate), respectively, in colloidal drug delivery systems (for example, liposomes, albumin microspheres, microemulsions, nano-particles and nanocapsules), in macroemulsions or precipitated or immobilized onto carriers or surfaces, for example by pcmc technology (protein coated microcrystals). Such techniques are disclosed in Remington: The Science and Practice of Pharmacy, 21st edition, Hendrickson R. Ed.

De-methylating agents like decitabine and 5-azacytidine can be used for the purposes of the present invention in any form and dosage as approved by the suitable drug regulatory agencies (e.g. FDA and EMA) for the treatment of cancer and MDS and as available on the market.

The CD33 antibody is typically formulated as infusion solution for intravenous application. As a typical example CD33-1 is formulated as an aqueous infusion solution (for being filled into glass vials) containing the following constituents at the following concentrations:

| | | | | |
|---|---|---|---|---|
| CD33-1 | 0.067 | mmol/l | 10.0 | g/l |
| Trisodium citrate dihydrate | 22.5 | mmol/l | 6.617 | g/l |
| Citric acid monohydrate | 2.5 | mmol/l | 0.525 | g/l |
| Sodium chloride | 125 | mmol/l | 7.305 | g/l |
| Polysorbate 20 (Tween 20) | | | 0.2 | g/l |
| Water for injection (WFI) | ad 1 | liter | | |

Also other suitable infusion formulations known in the art can be used.

The amount of the pharmaceutical composition that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the pharmaceutical compositions will also depend on the route of administration, and the seriousness of the disease or disorder, and should be decided according to the judgment of the practitioner and each patient's circumstances.

The pharmaceutical compositions comprise an effective amount of a drug(s) or agent(s) such that a suitable dosage will be obtained. Typically, this amount is at least about 0.01% of a drug or agent by weight of the pharmaceutical composition. When intended for oral administration, this amount can be varied to range from about 0.1% to about 80% by weight of the pharmaceutical composition. In one aspect, oral pharmaceutical compositions can comprise from about 4% to about 50% of the active constituents by weight of the pharmaceutical composition. In yet another aspect, present pharmaceutical compositions are prepared so that a parenteral dosage unit contains from about 0.01% to about 2% by weight of the active constituents.

For intravenous administration, the pharmaceutical composition can comprise from about 1 to about 50 mg of a drug or agent per kg of the patient's body weight. In one aspect, the pharmaceutical composition can include from about 1, 1.5 or 2.5 to about 50 mg of a drug or agent per kg of the patient's body weight. In another aspect, the amount administered will be in the range from about 1, 1.5 or 2.5 to about 25 mg/kg of body weight of a drug or agent.

In some embodiments, the dosage administered to a patient is less than 0.1 mg/kg to about 50 mg/kg of the patient's body weight. (For conversion to mg/mm2, a BSA of 1.8 m2 and a body weight of 80 kg can be used.)

Alternatively, the pharmaceutical composition according to the present invention can also be administered as a “per patient” amount. Suitable doses are 0.1 mg to 2000 mg, preferably 1 mg to 1000 mg, preferably 5 mg to 500 mg.

As discussed herein, pharmaceutical compositions herein can be administered intravenously or subcutaneously to the patient on a schedule that is, for example, daily, weekly, biweekly, tri-weekly or monthly to the patient. For example, pharmaceutical compositions herein can be administered weekly, for a period of 2 to 10 weeks, typically 3-6 weeks. In some embodiments, the dosage regimen of the pharmaceutical compositions herein maintains a blood serum concentration of antibody at least 5 μg/ml or at least 10 μg/ml during the dosage cycle. The pharmaceutical compositions herein can be administered, for example, from 1-8, or more cycles. In some embodiments, pharmaceutical compositions herein are administered chronically to a subject.

By way of example, the invention includes a method of treating a cancer, such as myeloid leukemia, by administering 0.1 mg/kg to 50 mg/kg, for instance about 1.5-8 or 2.5-8 mg/kg, of a pharmaceutical composition herein weekly. This treatment can be usually be continued for about 1-3 months, typically about two months. In an embodiment, the dosing schedule is maintained until a reduction in blasts is noted. For example, dosing can be continued up to about 6 months. This treatment can be followed by a less frequent dosing schedule, involving for instance biweekly doses (or twice per month). This dosing schedule can be maintained 1, 2, 3, 4, 5, 6 months or more to maintain a reduction in blasts and/or a remission.

In some embodiments, a prophylactic agent can be administered with pharmaceutical compositions herein to minimize infusion reactions. Suitable prophylactic agents include, for example, methyl prednisolone, diphenyldramine, acetaminophen or other suitable agent. The prophylactic agent can be administered prior to or at about the same time as the pharmaceutical compositions herein.

The pharmaceutical compositions herein can be administered by any convenient route, for example, by infusion or bolus injection, by absorption through epithelial or mucocutaneous linings (e.g., oral mucosa, rectal and intestinal mucosa, etc.). Administration can be systemic or local. Various delivery systems are known, e.g., encapsulation in liposomes, microparticles, microcapsules, capsules, etc., and can be used to administer the pharmaceutical compositions herein.

It can be desirable to administer the pharmaceutical compositions herein locally to the area in need of treatment, as appropriate for the drug or agent. This can be achieved, for example, and not by way of limitation, by local infusion during surgery; topical application, e.g., in conjunction with a wound dressing after surgery; by injection: by means of a catheter; by means of a suppository; or by means of an implant, the implant being of a porous, non-porous, or gelatinous material, including membranes, such as sialastic membranes, or fibers. In one embodiment, administration can be by direct injection at the site (or former site) of a cancer, tumor or neoplastic or pre-neoplastic tissue.

The pharmaceutical compositions herein can be delivered in a controlled release system, such as a pump or various polymeric materials. In yet another embodiment, a controlled-release system can be placed in proximity of the target of the pharmaceutical compositions herein, thus requiring only a fraction of the systemic dose (see, e.g., Goodson, in Medical Applications of Controlled Release, vol. 2, pp. 115-138, 1984). Other controlled-release systems discussed in the review by Langer (1990, Science 249: 1527-1533) can be used.

The pharmaceutical compositions herein are formulated in accordance with routine procedures as a pharmaceutical composition adapted for intravenous administration to animals, particularly human beings, as appropriate for the drug or agent. Typically, the carriers or vehicles for intravenous administration are sterile isotonic aqueous buffer solutions. Where necessary, the pharmaceutical compositions can also include a solubilizing agent. Pharmaceutical compositions for intravenous administration can optionally comprise a local anesthetic such as lignocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where drug or agent is to be administered by infusion, it can be dispensed, for example, with an infusion bottle containing sterile pharmaceutical grade water or saline. Where the drug or agent is administered by injection, an ampoule of sterile water for injection or saline can be provided so that the ingredients can be mixed prior to administration.

Pharmaceutical compositions of therapeutic agents also can be administered according to accepted dosage forms in the form of tablets, lozenges, aqueous or oily suspensions, granules, powders, emulsions, capsules, syrups, or elixirs, for example. Orally administered pharmaceutical compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the pharmaceutical compositions can be coated to delay disintegration and absorption in the gastrointestinal tract thereby providing a sustained action over an extended period of time. Selectively permeable membranes surrounding an osmotically active driving compound are also suitable for orally administered drugs or agents. In these later platforms, fluid from the environment surrounding the capsule is imbibed by the driving compound, which swells to displace the agent or agent pharmaceutical composition through an aperture. These delivery platforms can provide an essentially zero order delivery profile as opposed to the spiked profiles of immediate release formulations. A time-delay material such as glycerol monostearate or glycerol stearate can also be used.

The pharmaceutical composition can include various materials that modify the physical form of a solid or liquid dosage unit. For example, the pharmaceutical composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The pharmaceutical compositions can be administered to a patient in need thereof at a frequency, or over a period of time, that is determined by the attending physician. The pharmaceutical compositions can be administered over a period of 1 day, 2 days, 3 days, 5 days, 7 days, 10 days, 14 days, 21 days, 28 days, one month, two months, or longer periods of time. It is understood that the pharmaceutical compositions can be administered for any period of time between 1 day and two months or longer.

The combinations may be presented as a combined preparation kit. By the term “combined preparation kit” or “kit” as used herein is meant the pharmaceutical composition or compositions that are used to administer the pharmaceutical combinations according to the invention. When the active constituents of the pharmaceutical combinations, i.e. the CD33 antibodies and de-methylating agents and optionally the anti-neoplastic agent(s) are administered simultaneously, the combined preparation kit can contain each active constituent in a single pharmaceutical composition, such as a tablet, or in separate pharmaceutical compositions. When the active constituents are not administered simultaneously, the combined preparation kit will contain the active constituents in separate pharmaceutical compositions either in a single package or the active constituents in separate pharmaceutical compositions in separate packages or compartments.

In one aspect there is provided a pharmaceutical composition in the form of a combined preparation kit comprising

(i) a first compartment containing a first pharmaceutical composition comprising a the CD33 antibody;

(ii) a second compartment containing a second pharmaceutical composition comprising de-methylating agent; and optionally

(iii) a third compartment containing one or more pharmaceutical composition(s) comprising one or more additional anti-neoplastic agent(s).

In one embodiment there is provided a combined preparation kit comprising the active constituents as suitable pharmaceutical compositions, wherein the active constituents are provided in a form which is suitable for sequential, separate and/or simultaneous administration.

In one embodiment there is provided a combined preparation kit comprising the following components: a first container comprising a CD33 antibody as a suitable pharmaceutical composition; and a second container comprising an de-methylating agent as a suitable pharmaceutical composition, and a container means for containing said first and second containers.

The combination kit can also be provided by instruction, such as dosage and administration instructions. Such dosage and administration instructions can be of the kind that is provided to a doctor, for example by a drug product label, or they can be of the kind that is provided by a doctor, such as instructions to a patient.

In another aspect, the present invention also relates to CD33 antibodies for use in the treatment of cancer or MDS in combination with de-methylating agents.

In another aspect, the present invention relates to a method of treatment of cancer or MDS, comprising administration of a therapeutically effective amount of a CD33 antibody to a patient in need thereof, and furthermore comprising administration of a therapeutically effective amount of an de-methylating agent to the same patient within 72 hours before or after administration of said CD33 antibody. Worded differently, this aspect of the invention related to CD33 antibodies for use in the treatment of cancer or MDS in a patient, wherein one or more de-methylating agents are administered to the same patient within 72 hours before or after administration of said CD33 antibody.

In another embodiment the administration of the de-methylating agent is done within 36 hours before or after administration of said CD33 antibody.

In another embodiment the administration of the de-methylating agent is done within 24 hours before or after administration of said CD33 antibody.

In another embodiment the administration of the de-methylating agent is done within 12 hours before or after administration of said CD33 antibody.

In another embodiment the administration of the de-methylating agent is done within 6 hours before or after administration of said CD33 antibody.

In another embodiment the administration of the de-methylating agent is done within 3 hours before or after administration of said CD33 antibody.

In another embodiment the administration of the de-methylating agent is done within 2 hours before or after administration of said CD33 antibody.

In another embodiment the administration of the de-methylating agent is done within 1 hours before or after administration of said CD33 antibody.

In another embodiment the administration of the de-methylating agent is done within 30 minutes before or after administration of said CD33 antibody.

In another embodiment the administration of the de-methylating agent is done simultaneously with the administration of said CD33 antibody.

Simultaneous administration of the de-methylating agent and the CD33 antibody can typically be achieved by administering both de-methylating agent and CD33 antibody by simultaneous infusion out of separate infusion vessels, or by
Administering both de-methylating agent and CD33 antibody by simultaneous infusion out of the same infusion vessel, or by
Administering de-methylating agent orally while administering the CD33 antibody by infusion, or by
Administering de-methylating agent orally while administering the CD33 antibody subcutaneously.

In another embodiment herein the de-methylating agent is administered to the patient a significant time prior to administration of the CD33 antibody. Typically under this embodiment the de-methylating agent is administered to the patient 6 months, preferably 5 months, preferably 4 months, preferably 3 months, preferably 2 months, preferably 1 month, preferably 3 weeks, preferably 2 weeks, preferably 1 week prior to administration of the CD33 antibody.

### EXPERIMENTAL PART

### Acronyms and Abbreviations

ADCC antibody dependent cellular cytotoxicity

AML acute myeloid leukemia

ATCC American type culture collection

BSA bovine serum albumin

EC effective concentration

EDTA ethylenediaminetetraacetic acid

E:T effector to target cell ratio

Fc fragment crystallizable

FCS fetal calf serum

FITC fluorescein isothiocyanate

HBSS Hank's balanced salt solution

IgG immunoglobulin G

LDH lactate dehydrogenase

K_{D} equilibrium dissociation constant

MCB master cell bank

MEM minimal essential medium

MFI mean fluorescence intensity

PBMC peripheral blood mononuclear cell

PBS phosphate buffered saline

rpm revolutions per minute

### Material and Methods

The ability CD33 antibodies to mediate antibody-dependent cell-mediated cytotoxicity (ADCC) in the presence of effector cells pre-treated with demethylating agents is assessed using HL60 cells as target cells and human PBMCs as effector cells.

### Preparation of Target Cells (HL-60 Acute Myeloid Leukemia, DSMZ #ACC3)

An aliquot of the cell culture at a cell density between 1.5×10⁶/ml and 1.8×10⁶/ml and growing in the log-phase is centrifuged (200×g, i.e. 1000 rpm) for 10 min. Cells are washed once with washing medium (RPMI 1640 w/o L-glutamine) and pelleted (200×g, i.e. 1000 rpm; 10 min). Cell pellet is suspended in assay medium (1% BSA; AlbuMAX II (Gibco 11021-037)) and cell count is determined. Cell concentration is adjusted to 2×10⁵/ml.

### Preparation of Effector Cells

Approximately 100 ml whole blood drawn from healthy donors is used for the isolation of PBMC. 10 ml whole blood are diluted 1:3.6 with 26 ml HBSS (Gibco 14170-088; Paisley, Scotland) in a 50 ml tube. 18 ml diluted whole blood is layered on top of 12 ml Lymphoprep (Nycomed Pharma AS 1053980) in a 50 ml tube and centrifuged at 370×g (1400 rpm) for 35 min. The mononuclear cells from the interface are aspirated and washed first with HBSS (750×g, i.e. 1900 rpm; 10 min), then a second time with HBSS (300×g, i.e. 1200 rpm; 10 min) and finally with HBSS (160×g, i.e. 900 rpm; 10 min). The pelleted cells are gently suspended in culture medium/assay medium (10% heat-inactivated human AB serum in RPMI 1640 w/o L-glutamine) using a pipette and the cell count is determined in the cell counter. The PBMC concentration is adjusted to 1×10⁷/ml.

The freshly isolated PBMC (5×10⁵/m1) are pre-treated with de-methylating agents in culture medium (RPMI 1640 w/o L-glutamine supplemented with 10% human AB serum and decitabine or 5-azacytidine at a final concentration of 100 nM) in a tissue culture flask (75 cm²) at 37° C. in CO₂ incubator for 3 to 6 days. De-methylating agent treated PBMC are separated from cell debris on a Lymphoprep gradient. The purified de-methylating agent treated PBMC are suspended in culture medium/assay medium at a concentration of 1×10⁷/ml.

### Determination of Cytotoxicity

For all tested cell lines an E:T ratio of 25:1 is used. The co-cultivation of effector cells with target cells in presence of test antibody is performed in quadruplicates in 96-well round-bottom microtiter plates (Costar #3799) in a final volume of 200 μl assay medium per well consisting of 10% human AB serum and 1% BSA in RPMI in 1:1 ratio. In each experimental run two plates are assayed in parallel. First effector cells (PBMC cells in 100 μl 10% human AB serum in RPMI per well) are plated, followed by target cells (in 50 μl 1% BSA in RPMI per well) and antibody solution (candidate antibody diluted in 50 μl 1% BSA in RPMI). As a control, effector cells are cultivated in assay medium alone (effector cell control) and target cells are cultivated either in assay medium alone (spontaneous lysis) or in assay medium supplemented with 1% Triton X-100 (maximal lysis). The co-culture is incubated at 37° C. in a humid CO² incubator for 3 to 4 hours.

At the end of the incubation cells are removed from the culture medium by centrifugation (200×g, i.e. 1000 rpm; 10 min) at room temperature. Cell free supernatants (100 μl/well) are transferred into corresponding wells of a 96-well flat-bottom plate (Costar #3595). To determine the LDH activity in these supernatants 100 μl reaction mixture (freshly mixed 250 μl catalyst with 11.25 ml dye solution) are added to each well and incubated 30 min at room temperature in the dark. Then the absorbance is measured as described below.

Cytotoxicity Detection Kit (LDH Roche #11 644 793 001) is used to determine ADCC activity. The assay is based on the measurement of LDH enzyme activity released from plasma membrane-damaged cells. LDH released into the culture supernatants reduces the tetrazolium salt from the kit to formazan. The absorption maximum of formazan dye is measured at 490 nm against a reference wavelength of 650 nm in an ELISA plate reader.

To calculate percent cell mediated cytotoxicity five controls are performed in each experimental setup.
Background control I (1): LDH activity contained in the assay medium, which is subtracted from values (3) and (5).
Background control II (2): LDH activity contained in 1% Triton-X100 in assay medium, which is subtracted from maximal LDH release values (4).
Spontaneous LDH release (3): LDH activity released from target cells alone.
Maximal LDH release (4): Maximum releasable LDH activity in the target cells.
Effector cell control (5): LDH activity released from effector cells only.

For determination of the percentage cell mediated cytotoxicity the absorbance of each data point is subtracted by background absorbance. These corrected values are substituted into the following equation to calculate ADCC (%):

(effector/target cell mix−effector cell control−spontaneous release)/(maximal release−spontaneous release):

EC₅₀, bottom and top of concentration response curves were calculated by nonlinear regression analysis (GraphPad Prism software). Maximal lysis was defined as top-bottom. For each cell line two plates with quadruplicate data points were assayed in parallel.

TABLE 2

| | | Decitabine | 5-Azacitidine |
|---|---|---|---|
| | untreated | 100 nM | 100 nM |
| Relative Cytolysis (%) | 100 | 98 | 107 |
| Standard deviation | 0 | 15 | 12 |

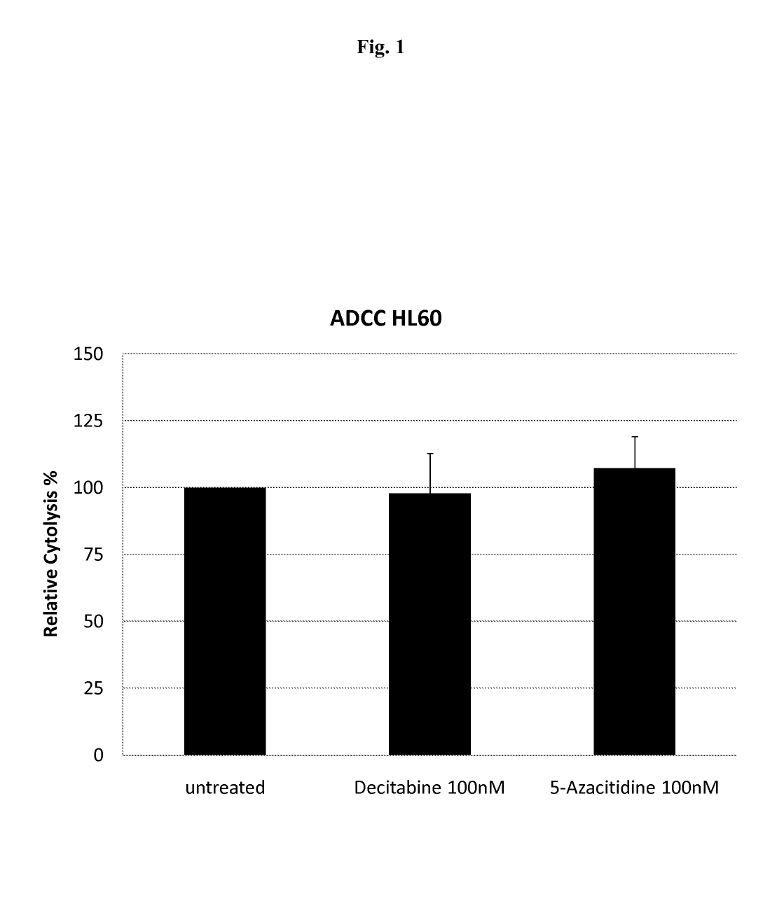

Results from 3 independent assays are shown in FIG. 1 and Table 2. Unexpectedly, treatment of effector cells with 5-azacytidine or decitabine at a concentration of 100 nM for 3 to 6 days did not significantly impact on ADCC of mAb 33-1 as can be seen by a relative cytolysis of 98% and 107%, respectively, as compared to cytolysis achieved with effector cells (PBMC) not treated with de-methylating agents. In particular, no significant decrease of cytolysis after treatment of effector cells with de-methylating agents was observed.

1. A pharmaceutical combination comprising one or more CD33 antibodies and one or more de-methylating agents.

2. The pharmaceutical combination according to claim 1, wherein the CD33 antibody has an immunoglobulin heavy chain with a CDR1 selected SeqID No: 1-14, a CDR2 selected from SeqID No: 15-28, a CDR3 selected from SeqID No: 29-42, and an immunoglobulin light chain with a CDR4 selected from SeqID No: 43-56, a CDR5 selected from SeqID No: 57-70 and a CDR 6 selected from SeqID No: 71-84.

3. The pharmaceutical combination according to claim 1, wherein the CD33 antibody has a heavy chain variable region selected from SeqID No: 85-98 and a light chain variable region selected from SeqID No: 99-112.

4. The pharmaceutical combination according to claim 1, wherein the CD33 antibody has an immunoglobulin heavy chain selected from SeqID No: 113-126 and an immunoglobulin light chain selected from SeqID No: 127-140.

5. The pharmaceutical combination according to claim 1, wherein the CD33 antibody has an immunoglobulin heavy chain according to SeqID No: 117 and an immunoglobulin light chain according to SeqID No: 131, optionally comprising one or more mutations in the Fc region.

6. The pharmaceutical combination according to claim 5, wherein the mutation is a double mutation S239D/I332E.

7. The pharmaceutical combination according to claim 1, wherein the de-methylating agent is selected from 5-azacytidine (azacitidine), 5-azadeoxycytidine (decitabine) and SGI-110.

8. The pharmaceutical combination according to claim 1, wherein the CD33 antibody has an immunoglobulin heavy chain according to SeqID No: 117 and an immunoglobulin light chain according to SeqID No: 131, optionally comprising one or more mutations in the Fc region, and wherein the de-methylating agent is 5-azacytidine.

9. The pharmaceutical combination according to claim 1, wherein the CD33 antibody has an immunoglobulin heavy chain according to SeqID No: 117 and an immunoglobulin light chain according to SeqID No: 131, optionally comprising one or more mutations in the Fc region, and wherein the de-methylating agent is 5-azadeoxycytidine (decitabine).

10. The pharmaceutical combination according to claim 8, wherein the mutation is a double mutation S239D/I332E.

11. The pharmaceutical combination according to claim 9, wherein the mutation is a double mutation S239D/I332E.

12. The pharmaceutical combination according to claim 1, further comprising one or more anti-neoplastic agents.

13. A pharmaceutical composition comprising the pharmaceutical combination according to claim 1 admixed with one or more pharmaceutically acceptable diluents and optionally one or more pharmaceutically acceptable agents.

14. A kit comprising:
(i) a first compartment containing a first pharmaceutical composition comprising a CD33 antibody having an immunoglobulin heavy chain with a CDR1 selected SeqID No: 1-14, a CDR2 selected from SeqID No: 15-28, a CDR3 selected from SeqID No: 29-42, and an immunoglobulin light chain with a CDR4 selected from SeqID No: 43-56, a CDR5 selected from SeqID No: 57-70 and a CDR 6 selected from SeqID No: 71-84;
(ii) a second compartment containing a second pharmaceutical composition comprising de-methylating agent; and optionally
(iii) a third compartment containing one or more pharmaceutical composition(s) comprising one or more anti-neoplastic agents.

15. A method of treatment of cancer or myelodysplastic syndrome, comprising administration of a therapeutically effective amount of a CD33 antibody to a patient in need thereof, and furthermore comprising administration of a therapeutically effective amount of a de-methylating agent to the same patient within 72 hours before or after administration of said CD33 antibody.

16. The method according to claim 15, wherein the CD33 antibody has an immunoglobulin heavy chain according to SeqID No: 117 and an immunoglobulin light chain according to SeqID No: 131, optionally comprising one or more mutations in the Fc region.

17. The method according to claim 16, wherein the mutation is a double mutation S239D/I332E.

18. The method of claim 15, wherein the de-methylating agent is selected from 5-azacytidine (azacitidine), 5-azadeoxycytidine (decitabine) and SGI-110.

19. The method of claim 15, wherein administration of the de-methylating agent is done within 36 hours, 24 hours, 12 hours, 6 hours, 3 hours, 2 hours, 1 hour or 30 minutes before or after administration of said CD33 antibody.

20. The method of claim 15, wherein administration of the de-methylating agent is done simultaneously with the administration of said CD33 antibody.

21. The method of claim 15, wherein the cancer is acute myeloid leukemia.

22. The method of claim 15, wherein the cancer is selected from non-small cell lung cancer, renal cell carcinoma, ovarian cancer, breast cancer, colorectal cancer and pancreatic cancer.


SEQUENCE LISTING (140 sequences)

SEQ ID NO: 1 - protein, 8 aa, Artificial
  CDR
NWADAFDI

SEQ ID NO: 2 - protein, 8 aa, Artificial
  CDR
NWADAFDI

SEQ ID NO: 3 - protein, 8 aa, Artificial
  CDR
HWLDAFDI

SEQ ID NO: 4 - protein, 8 aa, Artificial
  CDR
NWADAFDI

SEQ ID NO: 5 - protein, 8 aa, Artificial
  CDR
NWADAFDI

SEQ ID NO: 6 - protein, 8 aa, Artificial
  CDR
NWADAFDI

SEQ ID NO: 7 - protein, 8 aa, Artificial
  CDR
NWADAFDI

SEQ ID NO: 8 - protein, 19 aa, Artificial
  CDR
NWADAFDIWGQGTMVTVSS

SEQ ID NO: 9 - protein, 19 aa, Artificial
  CDR
NWADAFDIWGQGTMVTVSS

SEQ ID NO: 10 - protein, 10 aa, Artificial
  CDR
EGGVDWYFDL

SEQ ID NO: 11 - protein, 10 aa, Artificial
  CDR
EGGVDWYFDL

SEQ ID NO: 12 - protein, 10 aa, Artificial
  CDR
EGGVDWYFDL

SEQ ID NO: 13 - protein, 10 aa, Artificial
  CDR
EGGVDWYFDL

SEQ ID NO: 14 - protein, 10 aa, Artificial
  CDR
EGGVDWYFDL

SEQ ID NO: 15 - protein, 17 aa, Artificial
  CDR
RIIPILGVANYAQKFQG

SEQ ID NO: 16 - protein, 17 aa, Artificial
  CDR
RIIPIINIASYAQNFQG

SEQ ID NO: 17 - protein, 17 aa, Artificial
  CDR
RIIPIIGIANYAQKFQG

SEQ ID NO: 18 - protein, 17 aa, Artificial
  CDR
RIIPILGVADYAQKFQG

SEQ ID NO: 19 - protein, 17 aa, Artificial
  CDR
RIIPILGVADYAQKFQG

SEQ ID NO: 20 - protein, 17 aa, Artificial
  CDR
RIIPIVGIVNYAEKFQG

SEQ ID NO: 21 - protein, 17 aa, Artificial
  CDR
RVIPIIGIASYAQNFQG

SEQ ID NO: 22 - protein, 17 aa, Artificial
  CDR
RVIPIIGIASYAQNFQG

SEQ ID NO: 23 - protein, 17 aa, Artificial
  CDR
RVIPIIGIASYAQNFEG

SEQ ID NO: 24 - protein, 17 aa, Artificial
  CDR
RIIPILDMANYAQKFQG

SEQ ID NO: 25 - protein, 17 aa, Artificial
  CDR
RIIPIIGIVNYAQKFQG

SEQ ID NO: 26 - protein, 17 aa, Artificial
  CDR
RIIPILGMANYAQKFQG

SEQ ID NO: 27 - protein, 17 aa, Artificial
  CDR
RIIPIIGIVNYAQKFQG

SEQ ID NO: 28 - protein, 17 aa, Artificial
  CDR
RIIPILGIANYAQKFQG

SEQ ID NO: 29 - protein, 5 aa, Artificial
  CDR
DYAIS

SEQ ID NO: 30 - protein, 5 aa, Artificial
  CDR
SYAIS

SEQ ID NO: 31 - protein, 5 aa, Artificial
  CDR
SYAIS

SEQ ID NO: 32 - protein, 5 aa, Artificial
  CDR
DYAIS

SEQ ID NO: 33 - protein, 5 aa, Artificial
  CDR
DYAIS

SEQ ID NO: 34 - protein, 5 aa, Artificial
  CDR
SYAIS

SEQ ID NO: 35 - protein, 5 aa, Artificial
  CDR
SYAIS

SEQ ID NO: 36 - protein, 5 aa, Artificial
  CDR
SYAIS

SEQ ID NO: 37 - protein, 5 aa, Artificial
  CDR
SYAIS

SEQ ID NO: 38 - protein, 5 aa, Artificial
  CDR
SYAIS

SEQ ID NO: 39 - protein, 5 aa, Artificial
  CDR
SYAIS

SEQ ID NO: 40 - protein, 5 aa, Artificial
  CDR
SYAIS

SEQ ID NO: 41 - protein, 5 aa, Artificial
  CDR
SYAIS

SEQ ID NO: 42 - protein, 5 aa, Artificial
  CDR
SYAIS

SEQ ID NO: 43 - protein, 8 aa, Artificial
  CDR
QQFNSSIT

SEQ ID NO: 44 - protein, 8 aa, Artificial
  CDR
QQFNSSIT

SEQ ID NO: 45 - protein, 8 aa, Artificial
  CDR
QQFNSSIT

SEQ ID NO: 46 - protein, 8 aa, Artificial
  CDR
QQFNSSIT

SEQ ID NO: 47 - protein, 8 aa, Artificial
  CDR
QQFDSSIT

SEQ ID NO: 48 - protein, 8 aa, Artificial
  CDR
QQFNSSYT

SEQ ID NO: 49 - protein, 8 aa, Artificial
  CDR
QQFNSSIT

SEQ ID NO: 50 - protein, 8 aa, Artificial
  CDR
QQFDSSIT

SEQ ID NO: 51 - protein, 8 aa, Artificial
  CDR
QQFNSSIT

SEQ ID NO: 52 - protein, 8 aa, Artificial
  CDR
QQFNSSIT

SEQ ID NO: 53 - protein, 8 aa, Artificial
  CDR
QQFNSSIT

SEQ ID NO: 54 - protein, 8 aa, Artificial
  CDR
QQFNSSIT

SEQ ID NO: 55 - protein, 8 aa, Artificial
  CDR
QQFNSSIT

SEQ ID NO: 56 - protein, 8 aa, Artificial
  CDR
QQFNSSIT

SEQ ID NO: 57 - protein, 7 aa, Artificial
  CDR
DASSLES

SEQ ID NO: 58 - protein, 7 aa, Artificial
  CDR
DASSLES

SEQ ID NO: 59 - protein, 7 aa, Artificial
  CDR
DASSLES

SEQ ID NO: 60 - protein, 7 aa, Artificial
  CDR
DASSLES

SEQ ID NO: 61 - protein, 7 aa, Artificial
  CDR
DASSLES

SEQ ID NO: 62 - protein, 7 aa, Artificial
  CDR
DASRLES

SEQ ID NO: 63 - protein, 7 aa, Artificial
  CDR
AASSLES

SEQ ID NO: 64 - protein, 7 aa, Artificial
  CDR
AASSLES

SEQ ID NO: 65 - protein, 7 aa, Artificial
  CDR
AASSLES

SEQ ID NO: 66 - protein, 7 aa, Artificial
  CDR
DASSLES

SEQ ID NO: 67 - protein, 7 aa, Artificial
  CDR
DASSLES

SEQ ID NO: 68 - protein, 7 aa, Artificial
  CDR
DASSLES

SEQ ID NO: 69 - protein, 7 aa, Artificial
  CDR
DASSLES

SEQ ID NO: 70 - protein, 7 aa, Artificial
  CDR
DASSLES

SEQ ID NO: 71 - protein, 11 aa, Artificial
  CDR
RASQGISSVLA

SEQ ID NO: 72 - protein, 11 aa, Artificial
  CDR
RASQGISSALA

SEQ ID NO: 73 - protein, 11 aa, Artificial
  CDR
RASQGISSALA

SEQ ID NO: 74 - protein, 11 aa, Artificial
  CDR
RASQGISSVLA

SEQ ID NO: 75 - protein, 11 aa, Artificial
  CDR
RASQGISSVLA

SEQ ID NO: 76 - protein, 11 aa, Artificial
  CDR
RASQGISSALA

SEQ ID NO: 77 - protein, 11 aa, Artificial
  CDR
RASQGISSVLA

SEQ ID NO: 78 - protein, 11 aa, Artificial
  CDR
RASQGISSVLA

SEQ ID NO: 79 - protein, 11 aa, Artificial
  CDR
RASQGISSVLA

SEQ ID NO: 80 - protein, 11 aa, Artificial
  CDR
RASQGISSALA

SEQ ID NO: 81 - protein, 11 aa, Artificial
  CDR
RASQGISSALA

SEQ ID NO: 82 - protein, 11 aa, Artificial
  CDR
RASQGISSALA

SEQ ID NO: 83 - protein, 11 aa, Artificial
  CDR
RASQGISSALA

SEQ ID NO: 84 - protein, 11 aa, Artificial
  CDR
RASQGISSALA

SEQ ID NO: 85 - protein, 117 aa, Artificial
  VH
QVQLVQSGAEVKKPGSSVKVSCKASGGTFSDYAISWVRQAPGQGLEWMGRIIPILGVANYAQKFQGRVTITADKSTRTAYMELSSLRSEDTAVYYCARNWADAFDIWGQGTMVTVSS

SEQ ID NO: 86 - protein, 117 aa, Artificial
  VH
QVQLVQSGAEVKKPGSSVKVSCKASGDTFSSYAISWVRQAPGQGLEWMGRIIPIINIASYAQNFQGRVTITADKSTNTAYMELSSLRSEDTAVYYCARNWADAFDIWGQGTMVTVSS

SEQ ID NO: 87 - protein, 117 aa, Artificial
  VH
QVQLVQSGTEVKKPGSSVKVSCKVSGDTFSSYAISWVRQAPGQGLEWMGRIIPIIGIANYAQKFQGRVTITADKSTSTAYMELSSLRSEDTAVYYCARHWLDAFDIWGQGTMVTVSS

SEQ ID NO: 88 - protein, 117 aa, Artificial
  VH
QVQLVQSGAEVKKPGSSVKVSCKASGGTFSDYAISWVRQAPGQGLEWMGRIIPILGVADYAQKFQGRVTITADKSTRTAYMELSSLRSEDTAVYYCARNWADAFDIWGQGTMVTVSS

SEQ ID NO: 89 - protein, 117 aa, Artificial; identical to SEQ ID NO: 88
  VH

SEQ ID NO: 90 - protein, 117 aa, Artificial
  VH
QVELVQSGAEVQKPGSSVKVSCKASGDTFSSYAISWVRQAPGQGLEWMGRIIPIVGIVNYAEKFQGRVTITADKSMSTAYMELSSLRSEDTAVYYCARNWADAFDIWGQGTMVTVSS

SEQ ID NO: 91 - protein, 117 aa, Artificial
  VH
QVQLVQSGAEVKKPGSSVKVACKASGDTFSSYAISWVRQAPGQGLEWMGRVIPIIGIASYAQNFQGRVTITADRSTSTAYMALSSLRSEDTAVYYCARNWADAFDIWGQGTMVTVSS

SEQ ID NO: 92 - protein, 117 aa, Artificial; identical to SEQ ID NO: 91
  VH

SEQ ID NO: 93 - protein, 117 aa, Artificial
  VH
QVQLVQSGAEVKKPGSSVKVACKASGDTFSSYAISWVRQAPGQGLEWMGRVIPIIGIASYAQNFEGRVTITADRSTSTAYMALSSLRSEDTAVYYCARNWADAFDIWGQGTMVTVSS

SEQ ID NO: 94 - protein, 119 aa, Artificial
  VH
QVELVQSGAEVKKPGSSVKVSCKASGGTFSSYAISWVRQAPGQGLEWMGRIIPILDMANYAQKFQGRVTITADKSTYTAYMELSSLRSEDTAVYYCAREGGVDWYFDLWGRGTLVTVSS

SEQ ID NO: 95 - protein, 119 aa, Artificial
  VH
QVELVQSGAEVKKPGSSVKVSCKASEDTFSSYAISWVRQAPGQGLEWMGRIIPIIGIVNYAQKFQGRVTITADKSTYTAYMELSSLRSEDTAVYYCAREGGVDWYFDLWGRGTLVTVSS

SEQ ID NO: 96 - protein, 119 aa, Artificial
  VH
QVELVQSGAEVKKPGSSVKVSCKASGGTFSSYAISWVRQAPGQGLEWMGRIIPILGMANYAQKFQGRVTITADKSTYTAYMELSSLRSEDTAVYYCAREGGVDWYFDLWGRGTLVTVSS

SEQ ID NO: 97 - protein, 119 aa, Artificial
  VH
QVQLVQSGAEVKKPGSSMKVSCKASEDTFSSYAISWVRQAPGQGLEWMGRIIPIIGIVNYAQKFQGRVTITADKSTCTAYMELTSLRSEDTAVYYCAREGGVDWYFDLWGRGTLVTVSS

SEQ ID NO: 98 - protein, 119 aa, Artificial
  VH
QVQLVQSGAEVKKPGSSVKVSCKASGGTFSSYAISWVRQAPGQGLEWMGRIIPILGIANYAQKFQGRVTITADKSTYTAYMELSSLRSEDTAVYYCAREGGVDWYFDLWGRGTLVTVSS

SEQ ID NO: 99 - protein, 107 aa, Artificial
  VL
DIQLTQSPSSLSASVGDRVTITCRASQGISSVLAWYQQKPGKAPKLLIYDASSLESGVPSRFSGSGSGTDFTLTISSLQPEDFATYYCQQFNSSITFGQGTKLEIKR

SEQ ID NO: 100 - protein, 107 aa, Artificial
  VL
DIQLTQSPSSLSASVGDRVTITCRASQGISSALAWYQQKPGKAPKLLIYDASSLESGVPSRFSGSGSGTDFTLTISSLQPEDFATYYCQQFNSSITFGQGTKLEIKR

SEQ ID NO: 101 - protein, 107 aa, Artificial
  VL
DIQLTQSPSSLSASVGDRVTITCRASQGISSALAWSQQKPGKAPKLLIYDASSLESGVPSRFSGSGSGTDFTLTISSLQPEDFATYYCQQFNSSITFGQGTKLEIKR

SEQ ID NO: 102 - protein, 107 aa, Artificial; identical to SEQ ID NO: 99
  VL

SEQ ID NO: 103 - protein, 107 aa, Artificial
  VL
DIQLTQSPSSLSASVGDRVTITCRASQGISSVLAWYQQKPGKAPKLLIYDASSLESGVPSRFSGSGSGTDFTLTISSLQPEDFATYYCQQFDSSITFGQGTKLEIKR

SEQ ID NO: 104 - protein, 107 aa, Artificial
  VL
DIQLTQSPSSLSASVGDRVTITCRASQGISSALAWYQQKPGKAPKLLIYDASRLESGVPSRFSGSGSGTDFTLTISSLQPEDFTTYYCQQFNSSYTFGQGTKLEIKR

SEQ ID NO: 105 - protein, 107 aa, Artificial
  VL
DIQLTQSPSSLSASVGDRVTITCRASQGISSVLAWYQQKPGKAPKLLIYAASSLESGVPSRFSGSGSGTDFTLTISSLQPEDFATYYCQQFNSSITFGQGTKVEIKR

SEQ ID NO: 106 - protein, 107 aa, Artificial
  VL
DIQLTQSPSSLSASVGDRVTITCRASQGISSVLAWYQQKPGKAPKLLIYAASSLESGVPSRFSGSGSGTDFTLTISSLQPEDFATYYCQQFDSSITFGQGTKVEIKR

SEQ ID NO: 107 - protein, 107 aa, Artificial
  VL
DIQLTQSPSSLSASVGDRVTITCRASQGISSVLAWYQQKPGKAPKLLIYAASSLESGVPSRFSGSGSGTDFTLTISSLQPEDFATYYCQQFNSSITFGQGTKLEIKR

SEQ ID NO: 108 - protein, 107 aa, Artificial
  VL
DIQLTQSPSSLSASVGDRVTITCRASQGISSALAWYQQKPGKAPKFMIYDASSLESGVPSRFSGSGSGTDFTLTISSLQPEDFATYYCQQFNSSITFGQGTKLEIKR

SEQ ID NO: 109 - protein, 107 aa, Artificial; identical to SEQ ID NO: 108
  VL

SEQ ID NO: 110 - protein, 107 aa, Artificial; identical to SEQ ID NO: 108
  VL

SEQ ID NO: 111 - protein, 107 aa, Artificial; identical to SEQ ID NO: 108
  VL

SEQ ID NO: 112 - protein, 107 aa, Artificial; identical to SEQ ID NO: 108
  VL

SEQ ID NO: 113 - protein, 447 aa, Artificial
  VH
QVQLVQSGAEVKKPGSSVKVSCKASGGTFSDYAISWVRQAPGQGLEWMGRIIPILGVANYAQKFQGRVTITADKSTRTAYMELSSLRSEDTAVYYCARNWADAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 447 aa, Artificial
  VH
QVQLVQSGAEVKKPGSSVKVSCKASGDTFSSYAISWVRQAPGQGLEWMGRIIPIINIASYAQNFQGRVTITADKSTNTAYMELSSLRSEDTAVYYCARNWADAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 447 aa, Artificial
  VH
QVQLVQSGTEVKKPGSSVKVSCKVSGDTFSSYAISWVRQAPGQGLEWMGRIIPIIGIANYAQKFQGRVTITADKSTSTAYMELSSLRSEDTAVYYCARHWLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 447 aa, Artificial
  VH
QVQLVQSGAEVKKPGSSVKVSCKASGGTFSDYAISWVRQAPGQGLEWMGRIIPILGVADYAQKFQGRVTITADKSTRTAYMELSSLRSEDTAVYYCARNWADAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 447 aa, Artificial; identical to SEQ ID NO: 116
  VH

SEQ ID NO: 118 - protein, 447 aa, Artificial
  VH
QVELVQSGAEVQKPGSSVKVSCKASGDTFSSYAISWVRQAPGQGLEWMGRIIPIVGIVNYAEKFQGRVTITADKSMSTAYMELSSLRSEDTAVYYCARNWADAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 447 aa, Artificial
  VH
QVQLVQSGAEVKKPGSSVKVACKASGDTFSSYAISWVRQAPGQGLEWMGRVIPIIGIASYAQNFQGRVTITADRSTSTAYMALSSLRSEDTAVYYCARNWADAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 447 aa, Artificial; identical to SEQ ID NO: 119
  VH

SEQ ID NO: 121 - protein, 447 aa, Artificial
  VH
QVQLVQSGAEVKKPGSSVKVACKASGDTFSSYAISWVRQAPGQGLEWMGRVIPIIGIASYAQNFEGRVTITADRSTSTAYMALSSLRSEDTAVYYCARNWADAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 449 aa, Artificial
  VH
QVELVQSGAEVKKPGSSVKVSCKASGGTFSSYAISWVRQAPGQGLEWMGRIIPILDMANYAQKFQGRVTITADKSTYTAYMELSSLRSEDTAVYYCAREGGVDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 449 aa, Artificial
  VH
QVELVQSGAEVKKPGSSVKVSCKASEDTFSSYAISWVRQAPGQGLEWMGRIIPIIGIVNYAQKFQGRVTITADKSTYTAYMELSSLRSEDTAVYYCAREGGVDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 449 aa, Artificial
  VH
QVELVQSGAEVKKPGSSVKVSCKASGGTFSSYAISWVRQAPGQGLEWMGRIIPILGMANYAQKFQGRVTITADKSTYTAYMELSSLRSEDTAVYYCAREGGVDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 449 aa, Artificial
  VH
QVQLVQSGAEVKKPGSSMKVSCKASEDTFSSYAISWVRQAPGQGLEWMGRIIPIIGIVNYAQKFQGRVTITADKSTCTAYMELTSLRSEDTAVYYCAREGGVDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, Artificial
  VH
QVQLVQSGAEVKKPGSSVKVSCKASGGTFSSYAISWVRQAPGQGLEWMGRIIPILGIANYAQKFQGRVTITADKSTYTAYMELSSLRSEDTAVYYCAREGGVDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 213 aa, Artificial
  VL
DIQLTQSPSSLSASVGDRVTITCRASQGISSVLAWYQQKPGKAPKLLIYDASSLESGVPSRFSGSGSGTDFTLTISSLQPEDFATYYCQQFNSSITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 213 aa, Artificial
  VL
DIQLTQSPSSLSASVGDRVTITCRASQGISSALAWYQQKPGKAPKLLIYDASSLESGVPSRFSGSGSGTDFTLTISSLQPEDFATYYCQQFNSSITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 213 aa, Artificial
  VL
DIQLTQSPSSLSASVGDRVTITCRASQGISSALAWSQQKPGKAPKLLIYDASSLESGVPSRFSGSGSGTDFTLTISSLQPEDFATYYCQQFNSSITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 213 aa, Artificial; identical to SEQ ID NO: 127
  VL

SEQ ID NO: 131 - protein, 213 aa, Artificial
  VL
DIQLTQSPSSLSASVGDRVTITCRASQGISSVLAWYQQKPGKAPKLLIYDASSLESGVPSRFSGSGSGTDFTLTISSLQPEDFATYYCQQFDSSITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 213 aa, Artificial
  VL
DIQLTQSPSSLSASVGDRVTITCRASQGISSALAWYQQKPGKAPKLLIYDASRLESGVPSRFSGSGSGTDFTLTISSLQPEDFTTYYCQQFNSSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 213 aa, Artificial
  VL
DIQLTQSPSSLSASVGDRVTITCRASQGISSVLAWYQQKPGKAPKLLIYAASSLESGVPSRFSGSGSGTDFTLTISSLQPEDFATYYCQQFNSSITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 213 aa, Artificial
  VL
DIQLTQSPSSLSASVGDRVTITCRASQGISSVLAWYQQKPGKAPKLLIYAASSLESGVPSRFSGSGSGTDFTLTISSLQPEDFATYYCQQFDSSITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 213 aa, Artificial
  VL
DIQLTQSPSSLSASVGDRVTITCRASQGISSVLAWYQQKPGKAPKLLIYAASSLESGVPSRFSGSGSGTDFTLTISSLQPEDFATYYCQQFNSSITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 213 aa, Artificial
  VL
DIQLTQSPSSLSASVGDRVTITCRASQGISSALAWYQQKPGKAPKFMIYDASSLESGVPSRFSGSGSGTDFTLTISSLQPEDFATYYCQQFNSSITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 213 aa, Artificial; identical to SEQ ID NO: 136
  VL

SEQ ID NO: 138 - protein, 213 aa, Artificial; identical to SEQ ID NO: 136
  VL

SEQ ID NO: 139 - protein, 213 aa, Artificial; identical to SEQ ID NO: 136
  VL

SEQ ID NO: 140 - protein, 213 aa, Artificial; identical to SEQ ID NO: 136
  VL